(19) United States
(12) Patent Application Publication
(10) Pub. No.: US 2021/0204591 A1
(43) Pub. Date: Jul. 8, 2021

(54) TERPENES AND THC COMPOSITIONS AND METHODS

(71) Applicant: John V. Rivera, Vero Beach, FL (US)

(72) Inventor: John V. Rivera, Vero Beach, FL (US)

(21) Appl. No.: 17/248,047

(22) Filed: Jan. 6, 2021

Related U.S. Application Data
(60) Provisional application No. 62/958,335, filed on Jan. 8, 2020.
(60) Provisional application No. 62/957,359, filed on Jan. 6, 2020.

(51) Int. Cl. A24B 15/30; A61K 36/185; A24B 15/24; A24D 1/18

(52) U.S. Cl. CPC A24B 15/303; A24D 1/18; A24B 15/241; A61K 36/185

(57) ABSTRACT

In embodiments described herein, there is provided a composition is provided including 1) a terpene comprising at least one of: a) a primary terpene; b) a secondary terpene; and c) a tertiary terpene; 2) THC including a) synthetic THC, or b) organic THC, or a combination thereof; and optionally, 3) a natural smokable.

### CROSS-REFERENCE TO RELATED APPLICATIONS

This application claims the benefit of Provisional Appln. 62/957,359, filed Jan. 6, 2020, and claims the benefit of Provisional Appln. 62/958,335, filed Jan. 8, 2020, the entire contents of each of which are hereby incorporated by reference in their entirety.

### BACKGROUND

As the US increases the state-approved production and supply of cannabis derived pharmaceutical products, there are signs that synthetic cannabinoids will have advantages for the pharma industry over cannabinoids extracted from marijuana plants.

Noramco (Athens, Ga., US), a manufacturer of controlled substances, will add commercial-scale capacity at its Athens, Ga., US facility for dronabinol (tetrahydrocannabinol), a synthetic cannabinoid approved by the FDA as an anti-emetic in chemotherapy patients. Following implementation, Noramco will supply both dronabinol and cannabidiol from its facilities in the US and Europe. The expansion will scale up current clinical production capability to also include commercial production.

The move comes at the same time as a burgeoning federally regulated cannabinoid pharmaceutical market in the US. Earlier this year, the FDA approved GW Pharmaceuticals' (Cambridge, UK) Epidiolex, a cannabinoid for the treatment of seizures associated with Lennox-Gastaut syndrome or Dravet syndrome.

PharmSource, a Global Data product, previously reported this change would likely provoke a shake-up of controlled substance scheduling at the US Drug Enforcement Administration (DEA) that will decriminalize some cannabis based medicines, because a Schedule I designation currently held by marijuana implies the drug has no therapeutic value. Epidiolex was duly categorized as Schedule V in September 2018. This places it in the same Schedule as the anticonvulsant lacosamide and pregabalin, used for seizures, pain, and anxiety.

Production of cannabis-derived products is and will be a growing market. There is currently only a limited number of companies involved in the manufacture of these ultra-pure synthetic cannabinoids, such as Patheon (Durham, N.C., US), Dechra Pharmaceuticals (Northwich, UK), Pharmaceutics International (Hunt Valley, Md., US) and Procaps (Atlantico, Colombia).

Some cannabinoid players are banking on the supremacy of synthetic supply options. Noramco announced in October that it has filed a provisional patent application with the US Patent and Trademark Office (USPTO) directed to its pure Cannabidiol (CBD) synthesis.

Bill Grubb, Vice President of Business Development and Innovation, Noramco, stated, “Cannabidiol produced by Noramco has been independently verified to be more than 1,000 times lower than the recent FDA declaration of <0.1% THC in approved formulations; these ultra-low levels comfortably meet Canadian Health Authority requirements for natural health products.”

Wen-Chun Zhang, Senior Principal Scientist, Pharmaceutical API Process Development at Noramco, expressed his professional frustration about the prevailing bias for natural products over synthetic during a presentation on “The New Era of Ultra-Pure Synthetic Cannabinoids” on Nov. 6, 2018 at the American Association of Pharmaceutical Scientists (AAPS) meeting in Washington, D.C., US. Synthetic production is “way ahead cost-wise” when compared to other purifications and “there is a very good business case” for the synthetic pathway, Zhang said.

Synthesis avoids problems regarding spoilage of the marijuana plant, and the difficulties with the weight and transport of the controlled harvested product, he added. The GlobalData Pharma Intelligence Center Drugs Database shows over 130 cannabinoid drug products (excluding products in development for non-pharmaceutically regulated pathways) and will form an opportunity for cannabinoid companies in the near future.

The DEA restricts the production of controlled substances in the US. Noramco's synthesis pathway avoids these restrictions as the two necessary intermediates are not controlled substances, giving them another advantage over natural cannabis producers. The DEA's 2018 production quotas were 444 kg for cannabis, rising to 1,140 kg in August, and 385 kg for tetrahydrocannabinols (B/POR, September 2018). The proposed cannabis quota for 2019 has been increased further to 2,450 kg for research purposes; this may be a sign that the federal stance on the drug is beginning to align with that of individual states and that the federal government will eventually legalize medical marijuana.

Industry updates such as this are covered in the Bio/Pharma Outsourcing Reports from PharmSource. If you do not subscribe to PharmSource or the Bio/Pharmaceutical Outsourcing Report, please contact a GlobalData sales representative to gain access.

On Mar. 23, 2017, the DEA announced it has scheduled the newly approved synthetic THC-containing drug, Syndros, as a Class II substance. This announcement comes as a surprise to some for a number of reasons, including that the approved marketed cannabis-containing drug, Marinol, is a Schedule III, and the DEA clarified in December 2016 that all plant cannabis extracts continue to be classified as Schedule I, the same classification as heroin.

Schedule I drugs are defined as drugs with no currently accepted medical use and a high potential for abuse. Last year, DEA chief Chuck Rosenberg said the decision to continue to label cannabis as a Schedule I substance was rooted in science, relying heavily on the Food and Drug Administration's conclusion that marijuana has “no currently accepted medical use in treatment in the United States.” This is, of course, despite the FDA's approval of certain drugs like Marinol and now Syndros, which contain cannabis-derived active pharmaceutical ingredients. It is worth noting that following the FDA's approval of Syndros on Jul. 1, 2016, the FDA recommended to the DEA to schedule Syndros as a Schedule II drug.

Syndros uses a synthetic version of tetrahydrocannabinol (THC), the compound associated with the psychoactive effects of cannabis, unlike other cannabis extracts, such as Cannabidiol (CBD). THC and CBD are both natural elements of the cannabis plant. CBD has received special waivers for medical testing grants in relation to treating childhood epilepsy, while THC has been the subject of only two FDA-approved drugs, both using the same synthetic THC variant as Syndros, known as dronabinol.

As part of these actions, the DEA issued an interim final rule indicating that products containing delta-9-THC will be placed in Schedule II. Other cannabis-based drugs with dronabinol, such as Marinol, have been given a Schedule III status, which are defined as drugs with a moderate to low potential for physical and psychological dependence. The question investors pose is whether this Schedule II designation will make market penetration harder for the drug's manufacturer.

Marijuana advocates have long hoped that the DEA would lessen the classification of cannabis to a Schedule II substance, which are defined as drugs with a high potential for abuse, but are nonetheless available for prescription use and include drugs such as Vicodin, oxycodone, methamphetamine and Adderall. While Schedule II substances are still classified as dangerous, there are fewer restrictions on research, which remains an area where cannabis companies hope to make inroads as new states continue to propose bills to approve both the medical and recreational use of marijuana.

So what does this mean for the exploding growth of the cannabis industry? Some would say that the Schedule II classification is a step backward by the federal agencies in recognizing the medical value of cannabis. Others would argue that this is reflective of the current U.S. Attorney's position on the legalization of cannabis. Regardless, the Schedule II designation may make it more difficult for cannabis-containing drugs to achieve the market penetration the sponsors of those drugs desire. In addition, the real question is whether this will have a chilling effect on research and development of additional cannabis pharmaceuticals. There is clearly a need in the marketplace for increased testing and resources dedicated to monitoring quality, as well as marketing practices for cannabis companies.

### Background Terpenes

Terpenes are organic aromatic molecules that contribute to plants' odor. They are found in Cannabis plants and many other plant species. Each Cannabis strain consists of its own unique terpene profile which creates a distinguishable effect and smell. In total, there are over 130 terpenes that have been found in cannabis. There are 5 distinct classes of terpenes: 1) Monoterpenes, 2) Sesquiterpenes, 3) Flavonoids & Phenols, 4) Diterpene, and 5) Canabinoids.

### Definitions

### 1) Monoterpenes:

Are a class of terpenes that consist of two isoprene units and have the molecular formula C12H16. Monoterpenes may be linear or contain rings. Modified terpenes, such as those containing oxygen functionality or missing a methyl group are called monoterpenes.

### Beta-Myrcene—Terpene Profile

Myrcene has a pleasant sweet flavor and makes up about 65% of cannabis essential oil. Being one of the major terpenes in cannabis, Myrcene gives cannabis most of its earthy, musky and herbal scent. In thyme, Myrcene can be up to 40% of the weight of wild thyme leaves. B-Myrcene has sedative as well as motor relaxant effects. Found in Lemon Grass.

### Alpha-Pinene—Terpene Profile

Hinted in its name, a-Pinene is commonly found in coniferous trees, notably the pine. Has a very characteristic spicy, woody pine aroma. It is the most widely encountered terpene in nature and has a wide range of medicinal properties. Several studies revealed it as a strong bronchodilator, anti-inflammatory, antibacterial, sedative and anxiolytic. Found in Pine.

### Cis-Ocimene—Terpene Profile

As the second form of beta-ocimene, cis-beta-ocimene differs slightly from trans-beta-ocimene due to the location of its functional groups relative to the carbon chains contained within this type of monoterpene. Also found within a variety of plants and fruits ranging from orchids to mangoes and kumquats, including high concentrations in cannabis, cis-ocimene is characterized by its woodsy aroma and floral notes which contribute to its use in perfumes. Apart from its fragrance, cis-ocimene features several therapeutic benefits including decongestant properties along with antibacterial and antiseptic effects. Found in Black Currants.

### Beta-Curcumene—Terpene Profile

Beta-Curcumene is an exotic sesquiterpene found in wild turmeric. Like other terpenes derived from the turmeric plant, beta-curcumene's aromas of spice and ginger have made it a popular component in the fragrances and cosmetic industry. With a flavor profile of spice and slight bitterness, beta-curcumene is also often used to add a twist to both savory dishes and desserts. Over the centuries, beta-curumene has been hailed by traditional medicine practioners for the healing effect it can have on skin conditions, respiratory failure, and cardiovascular disease. Found in Tumeric.

### Terpinolene—Terpene Profile

The odor of the monoterpene Terpinolene is classified as terpy, pine, and lemon like. Other than its medicinal use, it's used in the plastic and resin production and is characterized by its light amber color. Moreover, Terpinolene can be found in some cannabis phenotypes at above 20% from total terpene content or not exist at all. When inhaled, it may have sedative properties. Found in Lime.

### Camphene—Terpene Profile

Camphene is a terpene typically found in certain vegetables, fruits and herbs including carrots, pepper, dill, fennel, nutmeg, thyme and more. This monterpene occurs most prominently in indica strains of cannabis. It is commonly used in fragrances and food flavor additive due to its minty and citrus characteristic and spicy notes. When applied, this terpene causes a cooling sensation. It is recognized primarily for its therapeutic properties which include anti-inflammatory, antibiotic, antioxidant, analgesic, and antifungal effects. Camphene is proven for its ability to fight infections while reducing inflammation and stress. Found in Sage.

### Trans-Ocimene—Terpene Profile

One of the two forms of beta-ocimene, trans-beta-ocimene is a monoterpene known for its woody and herbaceous aroma characteristics along with a sweet, floral and green flavor profile.

Ocimenes are consistently identified in higher concentrations across many cannabis strains relative to other terpene types. Found commonly in allspice, black currant, passion fruit, guava, and cinnamon bark, trans-ocimene is often used as a food additive or in perfumes thanks to their fragrant aroma. Aside from its use as a food additive and fragrance, trans-ocimene is known for containing several therapeutic properties including anti-inflammatory, antifungal, and antiviral effects. Found in Black Currants.

### Alpha-Terpinene—Terpene Profile

Alpha-Terpinene is commonly found in allspice and several essential oils including cardamom, marjoram, citrus, Eucalyptus, and Juniperus. Its woody, lemony and citrusy aroma contribute to its popular use as a fragrance and flavor compound. Apart from its use as a food additive due to its lemony flavor, on a therapeutic level, alpha-Terpinene is known for its rapid antioxidant properties along with sedative effects. Found in Lime.

### Piperitone—Terpene Profile

Commonly used in essential oils, Piperitone is a popular flavoring additive found in two forms referred to as D-form and L-form. Known for its herbaceous, comphoraceous aroma that also features fragrances of both peppermint and menthol, piperitone can also be characterized by its minty flavor and spicy notes along with a cooling feeling. Found in buchu, Pipertone is used in cosmetics and perfumes, with therapeutic properties that include uses as a natural diuretic and decongestant. Found in Mint.

### Eucalyptol—Terpene Profile

Eucalyptol is a widespread terpene naturally produced by the Eucalyptus tree but also in many other plants including laurel, rosemary, and citrus oils and juices. Known for its alluring aromatics of cardamom, spice, mint and pine, Eucalyptol has been shown to be effective in treating anti-inflammatory and advanced respiratory disorders. Research has demonstrated that the terpene can also alleviate conditions including cancer, memory problems, infections, gum disease joint pain. Thanks to its cooling, minty flavors, Eucalyptol is a common ingredient in gum, hard candies and toothpaste. Found in Eucalyptus.

### Linalool—Terpene Profile

Linalool occurs in over 200 species of plants, commonly in lavender, rose and citrus. This terpene usually constitutes 5% or less of cannabis essential oil and has pleasant floral notes. Nevertheless, Linalool exhibits strong biological activity. Research studies showed that Linalool is one of the most powerful sedating terpene upon inhalation in mice. Found in Lavender.

### Fenchyl Alcohol—Terpene Profile

Combining the sensory profiles of pine and citrus, fenchyl alcohol brings spicy, woody and slightly bitter notes to the food it flavors and refreshing, piney smells to the soaps, deodorants and fragrances it's incorporated in. Unsurprisingly, fenchyl alcohol can be found in the essential oils of citrus peel and eucalyptus but also in beer, brandy and fennel. Found in Basil.

### Beta-Pinene—Terpene Profile

One of the two isomers of pinene is B-Pinene. It is one of the most abundant compounds released by forest trees, especially pine trees. Furthermore, it can be efficiently absorbed through the skin, lungs and digestive system. This monoterpene is known for its antibacterial effects as well as antidepressant-like and sedative-like activity on mice. Found in Pine.

### Citronellol—Terpene Profile

Citronellol is a terpene that can be found in a variety of herbs and spices, including black pepper, lavender and chamomile, as well as in the essential oils of Buchu leaves and Mairehau roots. The rich aromas of rose, lemon and melon that characterize Citronellol have made it a mainstay ingredient of high-end fragrances. Though Citronellol may be best known for its insect-repelling capabilities, this terpene has an impressive track record of improving topical conditions. Moreover, as a diaphoretic, Cintronellol can aid digestive conditions and improve immune system operations. Found in Rose.

### Borneol—Terpene Profile

Borneol has a sharp piney odor and has a wide use in the cosmetics and drugs industry. It's been suggested that its burning taste is reminiscent of mint. Borneol has been identified in more than 250 essential oils from plants, herbs, leaves or bark and has been used in traditional Chinese medicine for thousands of years. Found in Cinnamon.

### Citronellal—Terpene Profile

Citronellal is a monoterpenoid. It is a colorless to a pale yellow liquid. As its name suggests, citronellal can be found in the essential oils of citrus fruits such as lemon, lime, and kumquat, and lemongrass. A fragrance staple thanks to its alluring blended aroma of rose and lemon. The terpene's citrusy flavor adds zest to desserts and candies. As a therapeutic agent, citronellal is primarily used to treat fungal infections. However, scientists are exploring additional use cases for citronellal, including but not limited to using citronellal for anti-inflammatory and antioxidant purposes. Found in Lemongrass.

### D/L-Fenchone

D-fenchone and L-Fenchone are the two enantiomers that make up the monoterpene Fenchone. D-Fenchone is found in the essential oils of Russian anise and fennel, as well as in thyme and licorice. L-Fenchone is derived from fennel and wormwood. Both the D and L forms of Fenchone have bracing flavors of citrus and camphor that are used to season beverages and desserts. D/L Fenchone's pervasive scents of mint and pine are in widespread use in soaps and shampoos, perfumes and cosmetics. The therapeutic benefits of Fenchone include the alleviation of skin contusions and the treatment of bacterial-diseases and fungal infections. Found in Pine.

### Geraniol—Terpene Profile

The isomer of nerol, geraniol is found in many essential oils, including rose and palmarosa. Prized by fragrance makers for its concentrated rose scent, the terpene alcohol is also a frequent component in aromatic candles, cosmetics and hygiene products. As a flavoring, geraniol is often used to reproduce the sweet, deep summer fruit tastes of plum and watermelon. The list of the medicinal properties of geraniol is extensive, with studies demonstrating the terpene's efficacy as an anthelmintic, antimicrobial, anti-inflammatory and anti-cancer compound. Found in Geranium.

### D-Limonene—Terpene Profile

With its sweet and citrusy odor, it is one of the most frequently used fragrances in cosmetic formulations. In addition, it's one of the most common terpenes in nature. It can be found in several food items but also in botanical insecticides and cleaning products. The medical properties of D-Limonene are well observed and include anti-inflammatory, antioxidant, antinociceptive, anticancer, antidiabetic, antihyperalgesic and antiviral effects. Found in Orange.

### 3-Carene—Terpene Profile

A bicyclic monoterpene, 3-Carene is found in the essential oils of basil, fennel, kumquat and lime. Thanks to its sweet and citrusy scent, 3-Carene is often included in soaps, creams, and perfumes. The monoterpene's piney, citrusy taste is used across the food and beverage industry to augment savory and sweet tastes. Early testing in guinea pigs has shown that 3-Carene may be beneficial to humans that are suffering from advanced respiratory diseases such as bronchitis, while other studies have shown 3-Carene to be effective against inflammation and fungal infections as well. Found in Angelica Root.

### Geranyl Acetate—Terpene Profile

Geranyl acetate is a monoterpene derived from a variety of herbs and vegetables, such as sassafras, geranium, lemongrass and carrot. Intense aromatics of lavender and flower are the reason geranyl acetate is used in so many hygienic products and perfumes. The monoterpene's concentrated flavors of spice and sweetness are used to enrich the taste of sweet and fruity desserts and candies. Scientists continue to discover new medicinal use cases for geranyl acetate, though the most recent findings point to geranyl acetate as beneficial in the fight against serious respiratory disorders, diabetes, malaria and bacterial diseases. Found in Carrot Seeds.

### Cuminaldehyde—Terpene Profile

As its name suggests, cuminaldehyde is produced in cumin but can also be found in cinnamon, parsley, and brandy. Cuminaldehyde is used for its cumin-like flavor primary in savory foods and seasonings, though it can be also be found in smaller concentrations in desserts. The exotic overtones of spice and cumin are responsible for cuminaldehyde's frequent presence in cosmetic products and perfumes. Like other compounds from the cumin family, cuminaldehyde is associated with a number of significant health benefits, including anti-cancer, anti-allergic, and anti-diabetic properties. Found in Cumin.

### Alpha-Phellandrene—Terpene Profile

Alpha-phellandrene is a one of two isomers of the monoterpene phellandrene. The compound can be found a large assortment of herbs, vegetables, and preserved foods, including cured pork, celery leaves, eucalyptus and dill. With an alluring scent mixture of pepper, mint, and fruit, alpha-phellandrene is a staple in perfumes and cosmetics. Its refreshing flavor of light citrus is why alpha-phellandrene is a go-to component of desserts and candies. As a therapeutic agent, alpha-phellandrene has been shown to be effective against liver cancer alongside known antibacterial and antioxidant qualities. Found in Cinnamon.

### Alpha-Thujone—Terpene Profile

One of two diastereomeric forms of thujone, alpha-thujone is produced in alcoholic beverages. The cedar-like, minty aroma of alpha-thujone is incorporated in popular fragrances and cosmetics. Savory dishes such as meat products and sauces are likely to include alpha-thujone thanks to the sage and spicy flavor it brings to the table. In addition to its widespread commercial use, alpha-thujone has been shown to exhibit medicinal benefits for inflammatory disorders and fungal infections. Moreover, the compound has been linked to successful treatment of Polycystic Ovary Syndrome (PCOC). Found in Mugwort.

### D/L-Menthol—Terpene Profile

Menthol is mainly produced from mint oils and is commonly used in the food and pharma industry. It has a peppermint-like aroma and creates a cooling sensation when applied to skin and mucosal surfaces. There is a difference in the molecular structure between D- and L-menthol, which can be simplified by thinking of these molecules as the mirror image of one another. D-menthol has less cooling effects than its “mirrored sibling”. Several medical properties of menthol are discovered such as antimicrobial, anesthetic and antidepressive effects. Found in Mint.

### Linalyl Acetate—Terpene Profile

Linalyl acetate is produced by a number of herbs and fruits, including lavender, bergamot, peach and sage. This terpene's rich scents of lavender and bergamot are responsible for its constant usage in fragrances and other cosmetics. A byproduct of linalool, linalyl acetate's enticing flavor blend of citrus, herbs and spice explain why linalyl acetate is used extensively in prepared desserts, beverages, and meat products. Clinical trials have demonstrated linalyl acetate's ability to alleviate anxiety disorders and treat inflammatory conditions. Found in Rose.

### Isopulegol—Terpene Profile

A monoterpene that naturally occurs in varied collection of fruits and herbs, including orange juice, mint, buchu oil, and lemon balm, isopulegol is characterized by its cooling, minty tastes which grace countless prepared desserts and beverages. These same aromatic nuances of peppermint and fruit have contributed to its frequent inclusion in hygienic products, cosmetics and perfumes. Isopulegol's list of therapeutic benefits is considerable. Modern medicine has discovered isopulgol's proficiency in combatting anti-inflammatory diseases, liver diseases, and as an absorption enhancer for transdermal treatment. Found in Lemongrass.

### Carvone—Terpene Profile

Primarily produced in the essential oils of caraway seeds and spearmint, Carvone has two forms: enantiomers d-carvone and l-carvone. d-Carvone takes on the aromatic properties of caraway, while 1-carvone has spearmint's characteristically minty smell. Both enantiomers are used as aromatic and flavoring agents, each figuring prominently in both savory and sweet foods. Documented health benefits of Carvone include easing digestion in adults and children and soothing young babies, while traditional medicine attributes menstrual blood flow stimulation and expectorant properties to the terpene as well. Found in Spearmint.

### Carvacrol—Terpene Profile

This exquisite monoterpenoid phenol is found in a smattering of herbs, including myrtle, thyme, oregano and lovage. With a warm, piquant aroma, Carvacrol is used to introduce intriguing notes into fragrances as well as to bring pain relief through aromatherapy. The monoterpenoid phenol's herbaceous roots lend it a depth of flavor that is utilized throughout the prepared food industry. Though research into Carvacrol's health benefits is ongoing, is has been linked to microbial properties and been found effective against food-borne diseases along with potential for use as cancer treatment. Found in Majoram.

### Gamma Terpinene—Terpene Profile

One of three terpineneisomers, this monoterpene is a significant component of citrus fruits but can also be found in a variety of herbs and essential oils. Y-Terpinene has a particularly beguiling aromatic blend of lime and tropical sweetness that is frequently utilized in fragrances. The monoterpene's lush, citrussy tastes are used as food flavorings in savory dishes, desserts and beverages. In recent years the medical community has uncovered several therapeutic benefits of γ-Terpinene, including antifungal qualities and antioxidant properties that have been linked to slowing the progression of diabetes and Alzheimer's disease. Found in Laurel Bay Leaves.

### Menthofuran—Terpene Profile

Like another monoterpene, Pulegone, Menthofuran is found in the essential oils of various minty herbs, including spearmint, peppermint, and pennyroyal. Perfumeries draw on Menthofuran's smells of nut and coffee in their creation of warm, tantalizing fragrances. The monoterpene's matching intense flavors of nut, coffee are commonly used to flavor desserts and hard candies. Found in Peppermint.

### Sabinene Hydrate—Terpene Profile

Sabinene hydrate is a monoterpene found in the oils of spearmint and peppermint, as well as in a selection of spicy herbs and citrus fruits. With notes of citrus and pine, sabinene hydrate's aroma profile is close to that of certain conifer terpenes. In addition to its use as a minty, refreshing scent in the fragrance industry, sabinene hydrate's winning flavor combination of citrus, wood and mint adds zest to beverages, desserts and candies. Research suggests that the monoterpene is effective against cancer but can also alleviate some of the more unpleasant side effects of chemotherapy without damaging its efficacy. Found in Thyme.

### Nerol—Terpene Profile

Nerol plays a significant role in determining the flavor and aroma profiles of rose and palmarosaoils, in which it can be found in large quantities. Thanks to its fragrant mixed rose and citrus aroma, the terpene is a common ingredient in the fragrances industry. With enticing tastes of fresh fruit and light floral touches, Nerol is often combined with other additives to create popular fruit flavors like raspberry and red apple. The terpene is used to treat sepsis along with bacterial infections and is also effective at soothing and healing topical conditions such as acne and minor contusions. Found in Tuberose.

### Sabinene—Terpene Profile

Sabinene is a terpene that's primarily produced in black pepper, the essential oil of carrot seeds and in specific varieties of oak and spruce. With its earthy roots, sabinene's enticing warm, spicy aroma is used in a variety of fragrances and aromatherapy products. The monoterpene's peppery flavor can be found sprinkled throughout most sectors of the processed food industry. Traditional medicine extolls sabinene's ability to boost the immune system, and recent medical research confirms sabinene's potency in fighting bacterial infections and cancer. Found in Turmeric.

### Thymol—Terpene Profile

The primary flavor component of its namesake, thyme, Thymol is also found in an abundance of fruits, herbs, and cheese, including blueberry, mango, licorice and Gruyere. With a forest taste profile of wood and spice along with a gentle, sweet aroma, Thymol lends itself well to hard candies and dessert. As a natural disinfectant, Thymol is an established healing agent in septic, fungal, and bacterial disorders. As such, the terpene is a key component in oral healthcare products such as toothpaste and mouthwash. Found in Thyme.

### Camphor—Terpene Profile

Camphor is a terpene that is distilled from the Camphor Tree, though it is also found in spiced fruit, vegetables and herbs such as raspberry, apricot and ginger. Thanks to its woody, minty flavors, Camphor is a common ingredient in desserts and beverages. Like other terpenes with a strong menthol-like aroma, Camphor is an expectorant and has been shown to improve congestion and respiratory conditions. That same aroma is what makes Camphor such an effective insect-repellant. Among the terpenes other documented therapeutic benefits are its anti-inflammation and anti-infection properties along with its ability to improve circulation and sex drive. Found in Rosemary.

### Pulegone—Terpene Profile

Pulegone is a monoterpene that is produced in plants such as peppermint and spearmint and is found in the essential oils of pennyroyal and blackcurrant. The terpene's cooling notes of peppermint and spearmint have made it a common dessert additive. With a bracing aromatic profile of peppermint and camphor, Pulegone is a frequent ingredient in toothpaste as well as fragrances. Pulegone is mucolytic and can help ease congestion and mild respiratory complaints. Other documented medicinal benefits of Pulegone include calming gastrointestinal distress, preventing ulcers and easing menstrual pain. Found in Orange.

### Bornyl Acetate—Terpene Profile

Bornyl Acetate is primarily produced from pine tree oils, although it also occurs naturally in other plants including thyme, rosemary and coriander. The terpene's heavy pine smell has made it popular with industries that appeal to the sense of smell, from fragrances to cleaning solutions. As mirrored images of each other, the d and l forms of the terpene have different molecular structures; l-Bornyl Acetate features a sweeter but milder flavor and aroma profile than its d twin. Medicinal properties associated with the terpene include anti-infection, anti-inflammatory and relaxation qualities. Found in Sage.

### Alpha-Terpineol—Terpene Profile

The monoterpene α-Terpineol is a major component of pine oils. Moreover, it's present in small amounts in many other essential oils such as eucalyptus and cajeput. It is one of five common isomers of terpineol. Besides its important role in the industrial field, α-Terpineol offers a wide range of medical properties as a local anesthetic, anticancer, anti-inflammatory and antihypertensive compound. Found in Lime.

### 2) Sesquiterpenes:

Sesquiterpenes are a class of terpenes that consist of three isoprene units and often have the molecular formula C₁₅H₂₄. Like monoterpenes, sesquiterpenes may be acyclic or contain rings, including many unique combinations. Biochemical modifications such as oxidation or rearrangement produce the related sesquiterpenoids.

Sesquiterpenes are found naturally in plants and insects, as semiochemicals, defensive agents or pheromones.

### M-Cymene—Terpene Profile

It is a constituent of a number of essential oils, most commonly the oil of cumin and thyme. Significant amounts are formed in sulfite pulping process from the wood terpenes. Found in Cumin.

### Cannabispiran—Terpene Profile

It is a constituent of a number of essential oils, most commonly the oil of cumin and thyme. Significant amounts are formed in sulfite pulping process from the wood terpenes. Found in Cannabis.

### Isocannabispiran—Terpene Profile

Isocannabispiran is one of thirty four noncannabinoid phenols that are known. Found in Cannabis.

### Beta-Farnesene—Terpene Profile

Beta-Farnesene is known to have anti-inflammatory, calming, antispasmodic, antibacterial, antifungal, and sedative properties. Its scent is characterized as a woody, green vegetative smell with a lavender background. Found in Ginger.

### Isoborneol—Terpene Profile

Isoborneol properties give stability to scent and has a distinctive healing scent, characterized by spicy, woody aroma. Used in many quality commercial products. Found in Camphor.

### Cis-Citral—Terpene Profile

Citral has a strong citrus scent. Neral's lemon odor is less intense, but sweeter. This small molecule is therefore a relaxing aroma used in perfumery for its citrus effect. Citral is also used as a flavor and for fortifying lemon oil. It also has strong anti-microbial qualities, and pheromonal effects in insects. Found in Carrot.

### Beta-Caryophyllene—Terpene Profile

Characterized by its spicy and peppery odor. This sesquiterpene is most commonly found in cloves, black pepper, basil, oregano and cannabis. There are 3 isomers (A, B, and Y-caryophyllene) in nature, however B-Caryophyllene is the most frequently encountered and most abundant of the isomers. In addition, as an outstanding feature, B-Caryophyllene is suggested to bind with CB2 receptors and activate the endocannabinoid system. B-Caryophyllene does not produce a psychoactive effect. Found in Black Pepper.

### Ledene—Terpene Profile

A naturally-occurring sesquiterpene of the Australian Tea Tree's essential oil, ledene is also found in small quantities in the essential oils of certain myrtle plants, sticky cassinia, and mint bush. Though ledene is not safe for consumption, the sesquiterpene's light, minty aroma has made it a standard component of cosmetics and soaps. In aromatherapy, ledene is lauded for its ability to boost the body's immune response and mitigate respiratory disorders. However, ledene is equally prized for its ability to relieve skin conditions like contusions and burns. Found in Tea Tree.

a-Curcumene—Terpene Profile

a-Curcumene is an herbaceous terpene found in rosemary and in high concentrations in the essential oils of ginger and turmeric. Curcumene belongs to the Sesquiterpene family. As an isomer of curcumene, α-curcumene plays a significant role in creating the turmeric spice's nutrient-rich, exotic flavor profile. Long before the advent of modern medicine, α-curcumene figured largely into traditional medicine in the form of turmeric-based cures for gastrointestinal discomfort, tumors and cardiovascular disorders. More recent scientific research has established α-curcumene's potency in treating rheumatic conditions as well as inflammation of the digestive tract. Found in Rosemary.

### Alpha-Bisabolol—Terpene Profile

The primary source of this colorless sesquiterpene is German chamomile. Therefore it has a pleasant light floral odor and has been shown to have anti-nociceptive and anti-inflammatory activities. Furthermore, it has been shown to help improve sleep quality. Terpene a-Bisabolol is well suited for use in skin care products and can be useful in both sensitive skin and child/baby products as a soothing ingredient. Found in Chamomile.

### Alpha-Humulene—Terpene Profile

The name Humulene comes from the plant where it was first extracted from, Humulus lupulus, also known as hops. Humulene can be up to 40% of the hops essential oil. In addition, its oxidation products in the brewing process give many beers their pleasant hoppy aroma. It has been shown to have antitumor activities. Found in Hops.

### Alpha-Gurjunene—Terpene Profile

Alpha-Gurjunene is found in some species of pearly everlasting and the thistle family of plants. With a spicy, woody aroma, Alpha-Gurjunene is often used to enhance fresh-smelling fragrances and cosmetic products. Similarly, its deep notes of spice and wood are used to flavor a variety of savory and dessert foods. The research community is continually discovering new therapeutic properties of Alpha-Gurjunene. The sesquiterpene has to date been shown to be valuable when employed against cancer and inflammation, is effective against ticks and can be used to dull pain. Found in Anophalis Nubigena.

### Trans-Nerolidol—Terpene Profile

Trans-Nerolidol is one of the two isomers that make up the Nerolidol sesquiterpene. This sesquiterpene is found in many aromatic herbs and plants, including ginger and jasmine, as well as in the essential oil neroli from which it takes its name. The terpene's fresh, forest scents of rose and citrus are used in cosmetics and household products, and its fresh, earthy taste makes it a common flavoring ingredient. Trans-Nerolidol's medicinal qualities are also well-documented. Recent research highlights Trans-Nerolidol's success in combating parasite-borne diseases and parasites, fungal infections, and in aiding antibiotics against bacterial infections. Found in Neroli.

### Caryophyllene Oxide—Terpene Profile

Caryophyllene Oxide has a sweet, woody aroma and is mainly found in lemon balm and geranium. It is the product of the oxidation of Caryophyllene. Furthermore, it's the component responsible for cannabis identification by drug-sniffing dogs. It is believed to be the only terpene in cannabis that has the ability to successfully bind with CB2 receptor. This unique skill makes it highly beneficial for medicinal uses, However, Caryophyllene Oxide only has a few known medical benefits. Found in Black Pepper.

### Beta Cedrene—Terpene Profile

Along with a-Cedrene, B-Cedrene is one of two isomers that form Cedrene, a sesquiterpene derived from the essential oils of cedar. Like α-Cedrene, B-Cedrene's crisp scents of pine and wood have contributed to its widespread use in fragrances, cosmetics, while its gentle sweet taste has seen it incorporated in beverages, sweeteners and baked goods. Similar to its isomer twin, B-Cedrene is a valued compound in traditional and holistic medicine. Studies have confirmed its therapeutic potency as anti-inflammatory, antiseptic, and diuretic, among its many medicinal qualities. Cedrene is still being researched for its effects on testosterone and fat loss, but preliminary evidence is promising. Found in Pine.

### Valencene—Terpene Profile

Named for the Valencia oranges in which it is found in high concentrations, Valencene is also produced in the peel and juice of many citrus plants and within other fruits and herbs. With its refreshingly sweet scents of citrus and wood, Valencene is a widely used component in fragrances, cosmetics and personal hygiene products. The terpene's strong citrusy flavor is routinely added to desserts, dairy products and breakfast cereals. As a bronchodilator, Valencene can ease respiratory conditions, and recent research has demonstrated the terpene's effectiveness against dermatological inflammations such as atopic dermatitis. Found in Orange.

### Alpha Cedrene—Terpene Profile

a-Cedrene is one of two isomers that make up Cedrene, a sesquiterpene that, along with Cedrol and Cedrenol, constitutes the sensory profile of the essential oils of cedar. This isomer's bracing aromas of fresh wood and pine have made it a common component of fragrances, cosmetics and hygienic products, while its mildly sweet taste has turned α-Cedrene into a recurring dessert and beverages ingredient. Used by holistic medicine practitioners as an antiseptic and mucolytic agent, recent scientific research suggests that α-Cedrene could be effective against tumors and bacterial infections. Found in Pine.

### Thujopsene—Terpene Profile

Along with Cedrol, Thujopsene is one of the main components of the varied essential oils of conifers. Thujopsene's woody, fresh aromas greatly contribute to these oils' use in aromatherapy as a soothing and even sedative agent. Frequently associated with other warm pine and citrus aromas popular in the cold winter months, Thujopsene is a common ingredient in hygiene products such as deodorant and shampoo as well as fragrances. The terpene is often used to combat topical conditions thanks to its anti-fungal, anti-septic and anti-inflammatory properties, though as an expectorant it is also effective against mild respiratory disorders. Found in Canary Island Juniper.

### Alpha-Farnesene—Terpene Profile

One of six Farnesene sesquiterpenes, α-Farnesene can be found in the peel of apples as well as in the fruit and essential oils of lemon and lime. Its alluring flavor profile blend of berry, herbal green and flowery notes explain a-Farnesene's inclusion in the ingredient list of savory and sweet products from all across the food industry. α-Farnesene has been shown to be very effective against inflammation and is known for its ability to alleviate stress and muscle spasms. Recent research additionally points to α-Farnesene as an integral compound in stopping tooth decay. Found in Jasmine.

### Cis-Nerolidol—Terpene Profile

Cis-Nerolidol, one of two Nerolidol isomers, is found in many essential oils, fruit, vegetables and herbs, including in its namesake, Neroli citrus oil. This terpene's fresh wood and fruity tones contribute to its extensive use as a dessert flavoring agent, as well as an aromatic in fragrances and hygiene products. The therapeutic benefits of Cis-Nerolidol are widespread and well-documented, including the inhibition of tumors and fungus as well as possessing antioxidant and antibacterial qualities. Other suggested medicinal use cases of Cis-Nerolidol include inducing drowsiness and soothing topical conditions. Found in Neroli.

### Cedrol—Terpene Profile

Cedrol is a musky terpene that is present in Juniper and other essential conifer oils, as well as in some herbs. This terpene's floral, woody aromas have made it a staple of hygiene and fragrance products, with both industries eager to capitalize on Cedrols lush forest perfume. Homeopathic medicine has long regarded Cedrol as an important healing compound thanks to its many demonstrated medicinal properties. In addition to serving as a sedative, Cedrols antispasmodic and astringent properties respectively relax muscles and sooth irritated skin. Like another conifer terpene, Thujopsene, Cedrol also has antiseptic, anti-fungal and anti-inflammatory qualities. Found in Cypress.

### Guaiazulene—Terpene Profile

A derivative of Azulene, Guaiazulene is found in the essential oils of German chamomile and guaiac. Marked by its aromatics of rose and sandalwood, Guaiazulene is a much sought-after ingredient of fragrances and high-end soaps. Traditional medicine has long considered Guaiazulene to be one of the more potent terpenes of healing, and modern medicine has begun to confirm Guaiazulene's immense psychological and physiological benefits. From an emotional perspective, Guaiazulene has been shown to boost feelings of happiness and peacefulness. The terpene's long list of physiological benefits include analgesic, anti-inflammatory, and nervine properties. Found in Chamomile.

### Farnesol—Terpene Profile

Farnesol is a terpene alcohol that can be found in over 30 essential oils, with an especially high concentration in the tropical Cabrueva oil. Thanks to its summery fruit flavors of peach, apricot and grapes, Farnesol is a frequent addition to desserts and candies, while its light aromatic blend of fruit and flowers have made it a key component of perfumery. The terpene has been clinically linked to the prevention of different types of cancer, in addition to exhibiting anti-inflammatory, antibacterial and antispasmodic properties. Found in Jasmine.

### Cuparene—Terpene Profile

Primarily produced by the Atlantic White Cypress tree, this sesquiterpene can also be found in the downy Japanese Enokitake mushroom. Like other conifer terpenes, Cuparene is characterized by its piney, woody aroma and its spicy, woody tang, for which it is used in desserts and chewing gum. Though the medicinal properties of Cuparene have not been explored as broadly as those of other conifer terpenes, Cuparene has been documented as being beneficial in the fight against blood cancer and for treating inflammation and bacterial infections. Found in Atlantic White Ceder.

### Isolongifolene—Terpene Profile

Isolongifolene is a derivative of Longifolene, a terpene produced in various strains of pine resin. Like other terpenes in the conifer family, Isolongifolene is characterized by woody and amber aromas and flavors that make it a prevalent ingredient in fragrances and cosmetics but also in tobacco and tea flavorings. The terpene shares many therapeutic benefits with other conifer-sourced terpenes, such as analgesic, anti-inflammatory and antispasmodic properties. Isolongifolene has also been shown to be beneficial for women's health, including in the prevention and treatment of breast cancer, menopause hormonal therapy and for boosting fertility. Found in Pine.

### Guaiol—Terpene Profile

Guaiol is a sesquiterpenoid alcohol primarily produced in the essential oil of its namesake Guaiacum and in the Cypress Pine. One of the Cannabis plant's most highly concentrated terpenes, the compound's heady pine, rose and violet aroma is the reason that Gauiol is frequently used in products that promote sensory experiences through smell, such as soaps and perfumes. A key therapeutic compound in traditional medicine's treatment of rheumatic diseases, modern medicine has additionally confirmed Guaiol's potent antifungal, antibacterial and diuretic properties. Found in Guaiacum.

### 3) Flavonoids and Phenols:

Natural phenolic compounds play an important role in cancer prevention and treatment. Phenolic compounds from medicinal herbs and dietary plants include phenolic acids, flavonoids, tannins, stilbenes, curcuminoids, coumarins, lignans, quinones, and others. Flavonoids are the largest group of naturally occurring phenolic compounds, which occurs in different plant parts both in free state and as glycosides. Glycosides are found in the plant genera Digitalis, Scilla, and Strophanthus. They are used in the treatment of heart diseases, e.g., congestive heart failure and arrhythmia.

### Phloroglucinol—Terpene Profile

Phloroglucinol is a weak triprotic acid. Found in Brown Alga.

### Cannabistilbene I—Terpene Profile

Found in Cannabis.

### Cannflavin C—Terpene Profile

Cannflavins are a group of chemical compounds found in Cannabis sativa. Chemically, they are flavonoids. Cannflavin-C, a non-psychoactive substance in the cannabis plant. Found in Cannabis.

### 2-Carene—Terpene Profile

2-Carene is a bicyclic monoterpene consisting of fused cyclohexene and cyclopropane rings. It occurs as a constituent of turpentine, Found in Allspice.

### Quercetin—Terpene Profile

Quercetin has multiple influences on immune system function, aside from its most commonly known mast cell stabilizing activity. Quercetin smells like rubber because it is a sulfur compound. Found in Red Onion.

### Isocannabispiran—Terpene Profile

Isocannabispiran is one of thirty-four noncannabinoid phenols that are known. Found in Cannabis.

### Alpha-Cannabispiran—Flavonoid Profile

Alpha-cannabispiranol is a byproduct of cannabis's secondary metabolism. It is a cannabinoid, as such it is only found in the cannabis plant. As is the case with other cannabinoids, the therapeutic properties of alpha-cannabisprianol are only now beginning to be researched in depth. Recent studies point to alpha-cannabispiranol as a potent treatment possibility for cancer and in particular, lymphoma. Found in Cannabis.

### Kaempferol—Flavonoid Profile

Kampferol is a natural flavonoid found in an assortment of fruit and vegetables, such as broccoli, blackberry and squash. As an antioxidant, kaempferol is often investigated for its remarkable effectiveness against different types of cancer, though the flavonoid has also shown promise in terms of therapeutic applications for cardiovascular, viral, and bacterial diseases. This small polyphenolic molecule has great shows great potential for healing. Found in Linden Flower.

### Dihydro-Resveratrol—Flavonoid Profile

Though it is found in cannabis, Dihydro-Resveratrol is produced in wine. Dihydro-Resveratrol has so far been found to be beneficial in fighting breast cancer and other cancers impacted by hormone-sensitive tumor cell lines. Found in Grapes.

### Vincenin-2—Flavonoid Profile

Vincenin-2 is a trihydroxyflavone that is found in nature in certain strains of the daisy family. Researchers have demonstrated Vincenin-2's potential to alleviate inflammation, to fight cancer and to treat diabetes. Found in Lavender.

### Cytisoside—Flavonoid Profile

Found in carnations, cytisoside is a flavonoid with concrete clinical applications. While the research on cytisoside is by no means exhausted, scientists have so far shown how cytisoside can be used to combat microbial infections, enteritis, and anuria. Found in Honeyberry.

### Chrysoeriol—Flavonoid Profile

Chrysoeriol has potential to regulate sugar levels in diabetes. It is s a bronchodilator, and effective for lowering blood pressure. Another derivative of luteolin, chrysoeriol is primarily found in alfalfa, though the flavone can be found in smaller quantities in other plants as well. Together with luteolin, chrysoeriol has demonstrated its potency against breast cancer and potential to regulate diabetes. Found in Yerba Santa.

### Luteolin-Glucoside—Flavonoid Profile

Like apigenin-glucoside, luteolin-glucoside can also be found in dandelion coffee, though this flavonoid is additionally present in the peach-leaved bellflower and artichoke plant. While modern medicine continues to assess cynaroside's therapeutic benefits and medicinal qualities, scientists have already discovered cynaroside's potency against inflammation and how it is able to help prevent edemas and ulcers. Found in Anise.

### Cannflavin A—Flavonoid Profile

A flavonoid unique to the cannabis plant, Cannflavin A's remarkable medicinal qualities have been well documented. In addition to its ability to inhibit pain, recent studies have indicated that Cannflavin A is beneficial for addressing rheumatic conditions and psychiatric disorders. Found in Cannabis.

### Vitexin—Flavonoid Profile

Vitexin is a trihydroxyflavone that is produced in bamboo leaves, pearl millet, and passion flower. The more promising studies on vitexin show the flavone may be useful in lessening inflammation, managing diabetes and preventing fertility issues. Vitexin further contributes to the reduction of neural cell death observed in neurodegenerative diseases. Although this review and other recent studies demonstrate its therapeutic effects, further research is needed to substantiate existing findings and accelerate knowledge in this area. Found in Passion Flower.

### Apigenin-Glucoside—Flavonoid Profile

Apigenin-glucoside rarely appears in nature and is primarily found in roasted dandelion roots and in strains of germanders. Among apigenin-glucoside's therapeutic qualities are inhibition of bacterial and respiratory tract infections. Moreover, it may be useful for fighting inflammation and alleviating pain. Found in Chamomile.

### Apigenin—Flavonoid Profile

This flavonoid is commonly associated with an assortment of herbs and vegetables, with concentrations found primarily in chamomile but also in gingko biloba leaf and celery. A natural antioxidant, apigenin is widely used in creams, hair-conditioner and other cosmetic products, and dietitians have begun to recommend the consumption of fruit and vegetables containing apigenin to lower the risk of cancer. The medicinal properties of apigenin have been widely documented. Not only has the flavonoid been shown to be effective against cancer, it may also benefit a wide range of neuro-psychological conditions such as amnesia, Alzheimer's and depression. Found in Chamomile.

### Cannflavin B—Flavonoid Profile

These two isomers are flavones that are unique to cannabis. Compared to Cannflavin A, this flavone attracted less attention in the past. Medical properties like anti-inflammatory activity can be deduced from the general research of Cannflavins. Found in Cannabis.

### Orientin—Flavonoid Profile

A derivative of luteolin, orientin is produced in bamboo leaves, found in buckwheat sprout and identifiable in several other herbs and vegetables. Over the centuries traditional medicine has turned to this flavone to solve a variety of ailments. Recent studies confirm orientin's therapeutic abilities for treating a wide range of disorders and conditions thanks to its antiviral, antibacterial, anti-inflammation, anti-depressant properties and more. Found in Bamboo.

### Luteolin—Flavonoid Profile

Named after the Reseda luteola plant from which it is derived, Luteolin is also found in herbs and green vegetables such as broccoli and celery. A vast amount of research on Luteolin has been conducted and there are current efforts underway to uncover more of its therapeutic capabilities. To date, Luteolin has been linked to antioxidant, anti-inflammatory, anti-cancer properties and can be helpful in reducing the risk and extent of cardiovascular conditions Found in Mullein.

### Isovitexin—Flavonoid Profile

This trihydroxyflavone is a derivative of apigenin. Found in cannabis, passionflower, buckwheat seed and a number of other plants, isovitexin can be used to treat inflammation. Isovitexin is also being examined as a potential healing agent for diabetes and Alzheimer's. Found in Passion Flower.

### 4) Diterpene:

Diterpenes are a class of chemical compounds composed of two terpene units, often with the molecular formula C20H32. Diterpenes consist of four isoprene subunits. They are biosynthesized by plants, animals and fungi via the HMG-CoA reductase pathway, with geranylgeranyl pyrophosphate being a primary intermediate. Diterpenes form the basis for biologically important compounds such as retinol, retinal, and phytol. They are known to be antimicrobial and anti-inflammatory.

### Phytol—Terpene Profile

Phytol is an acyclic diterpene alcohol that is found in small quantities in cannabis plants. Its odor is very faint and is described as floral. Phytol is used as a precursor for the manufacture of synthetic forms of vitamin E and vitamin K. Furthermore, a study from 2014 revealed anxiolytic-like effects of Phytol on mice. Found in Green Tea.

### 5) Cannabinoids:

A cannabinoid is one of a class of diverse chemical compounds that acts on cannabinoid receptors, which are part of the endocannabinoid system found in cells that alter neurotransmitter release in the brain. The endocannabinoid system (ECS) is a biological system composed of endocannabinoids, which are endogenous lipid-based retrograde neurotransmitters that bind to cannabinoid receptors, and cannabinoid receptor proteins that are expressed throughout the vertebrate central nervous system, including the brain and peripheral nervous system. The endocannabinoid is involved in regulating physiological and cognitive processes, including fertility, pregnancy, during pre and postnatal development, appetite, pain-sensation, mood, and memory, and in mediating the pharmacological effects of cannabis.

Two primary endocannabinoid receptors have been identified: CB1, first cloned in 1990; and CB2, cloned in 1993. CB1 receptors are found predominantly in the brain and nervous system, as well as in peripheral organs and tissues, and are the main molecular target of the endocannabinoid ligand (binding molecule), anandamide, as well as its mimetic phytocannabinoid, THC. One other main endocannabinoid is 2-arachidonoylglycerol (2-AG) which is active at both cannabinoid receptors, along with its own mimetic phytocannabinoid, CBD. 2-AG and CBD are involved in the regulation of appetite, immune system functions and pain management.

Ligands for these receptor proteins include the endocannabinoids produced naturally in the body by animals; phytocannabinoids, found in cannabis; and synthetic cannabinoids, manufactured artificially. The most notable cannabinoid is the phytocannabinoid tetrahydrocannabinol (THC), the primary psychoactive compound in cannabis. Cannabidiol (CBD) is another major constituent of the plant. There are at least 113 different cannabinoids isolated from cannabis, exhibiting varied effects.

### Cannabichromene

Found in cannabis.

### Carnagerol

Found in cannabis.

### Cannabinerolic Acid A (Z-CBGA-C5 A)

Found in cannabis.

### Cannabigerovarian (CBGV-C3)

Found in cannabis.

### Cannabigerol Monomethylether

Found in cannabis.

### Cannabigerolic Acid A/B

Found in cannabis.

### Cannbigerol

Found in cannabis.

### A 8 Tetrahydrocannabinolic Acid

Found in cannabis.

### Tran's A8-Tetrahydrocannabinol

Found in cannabis

### A8 Tetraydrocannabinol

Found in cannabis.

### Tetrahydrocannabinol-C4

Found in cannabis.

### Tetraydrocannabiorcolic Acid (A9 THCOA-C1)

Found in cannabis.

### Tetraydrocannabiorcol

Found in cannabis.

### Tetrahydrocannabivarin

Found in cannabis.

### Synthetic Cannabinoids:

Synthetic cannabinoids are a class of molecules that bind to the same receptors to which cannabinoids in cannabis plants THC and CBD attach. They are designer drugs, commonly sprayed onto plant matter and are usually smoked, although they have also been consumed in a concentrated liquid form in the United States and United Kingdom since 2016.

On Mar. 23, 2017, the DEA announced it has scheduled the newly approved synthetic THC-containing drug, Syndros, as a Class II substance. This announcement comes as a surprise to some for a number of reasons, including that the approved marketed cannabis-containing drug, Marinol, is a Schedule III, and the DEA clarified in December 2016 that all cannabis extracts from the cannabis plant continue to be classified as Schedule I, the same classification as heroin.

Marinol is approved by the FDA for medical uses, whereas marijuana has not been approved for any medical use at all at the federal level. Marinol also known as Dronabinol is a man-made form of cannabis (also known as marijuana). Additionally, Marinol, or dronabinol, does not contain all the components of the full marijuana plant. The medication has a synthetic version of THC (delta-9-tetrahydrocannabinol), one of over 60 cannabinoids found in Cannabis sativa. Two cannabinoids in marijuana have been studied for medicinal uses: THC and CBD.

Schedule I drugs are defined as drugs with no currently accepted medical use and a high potential for abuse. Last year, the DEA made a decision to continue to label cannabis as a Schedule I substance was rooted in science, relying heavily on the Food and Drug Administration's conclusion that marijuana has “no currently accepted medical use in treatment in the United States.” This is, of course, despite the FDA's approval of certain drugs like Marinol and now Syndros, which contain cannabis-derived active pharmaceutical ingredients. It is worth noting that following the FDA's approval of Syndros on Jul. 1, 2016, the FDA recommended to the DEA to schedule Syndros as a Schedule II drug.

Syndros uses a synthetic version of tetrahydrocannabinol (THC), the compound associated with the psychoactive effects of cannabis, unlike other cannabis extracts, such as cannabidiol (CBD). THC and CBD are both natural elements of the cannabis plant. CBD has received special waivers for medical testing grants in relation to treating childhood epilepsy, while THC has been the subject of only two FDA-approved drugs, both using the same synthetic THC variant as Syndros, known as dronabinol.

As part of these actions, the DEA issued an interim final rule indicating that products containing delta-9-THC will be placed in Schedule II. Other cannabis-based drugs with dronabinol, such as Marinol, have been given a Schedule III status, which are defined as drugs with a moderate to low potential for physical and psychological dependence. The question investors pose is whether this Schedule II designation will make market penetration harder for the drug's manufacturer.

Marijuana advocates have long hoped that the DEA would lessen the classification of cannabis to a Schedule II substance, which are defined as drugs with a high potential for abuse, but are nonetheless available for prescription use and include drugs such as Vicodin, oxycodone, methamphetamine and Adderall. While Schedule II substances are still classified as dangerous, there are fewer restrictions on research, which remains an area where cannabis companies hope to make inroads as new states continue to propose bills to approve both the medical and recreational use of marijuana.

### Hemp:

Hemp, or industrial hemp, is a strain of the Cannabis sativa plant species that is grown specifically for the industrial uses of its derived products. It is one of the fastest growing plants and was one of the first plants to be spun into usable fiber 10,000 years ago. It can be refined into a variety of commercial items, including paper, textiles, clothing, biodegradable plastics, paint, insulation, biofuel, food, and animal feed.

Although cannabis as a drug and industrial hemp both derive from the species Cannabis sativa and contain the psychoactive component tetrahydrocannabinol (THC), they are distinct strains with unique phytochemical compositions and uses. Hemp has very low concentrations of THC and higher concentrations of cannabidiol (CBD), which decreases or eliminates its psychoactive effects. The legality of industrial hemp varies widely between countries. Some governments regulate the concentration of THC and permit only hemp that is bred with an especially low THC content, less than 3%.

Hemp buds, also known as hemp flowers or CBD strains of cannabis. These hemp flowers are a brand new concept in the CBD market and will undoubtedly take some getting used to, but they're actually strains of the cannabis plant that contain less than 0.3% of THC, meaning you can roll and smoke these buds just like you would an actual joint, but you won't get high.

As an alternative to getting high, this concept was designed for those looking to relax and alleviate pain. These buds are taken straight from the Cannabis sativa plant, so while they look and smell exactly like traditional weed buds, these hemp flowers don't contain enough THC to actually get you high. These hemp buds contain a high concentration of cannabinoids, in comparison to the roots, stalk, and leaves of the cannabis plant. While other forms of CBD include oils, edibles, and tinctures, this brand new form of CBD offers yet another way to incorporate CBD into your daily habits.

CBD provides many of the same pain-relieving, calming, and healing properties as it is also derived from the Cannabis sativa plant. Now instead of gleaning buds that are rich in THC that can be rolled and smoked as a marijuana joint, there has been an increasing demand for buds that are rich in CBD, known as hemp flowers or hemp buds. These hemp buds are specifically grown to be rich in CBD, but low in THC, making it legal for public sale and consumption.

### SUMMARY

Embodiments of the present invention include various compositions of natural and synthetic THC, terpenes, and natural smokables. These compositions may be used in the treatment of various ailments, and may be formulated in many different formulations including smokables, tinctures, oils, vapes, chewing gum, patches, gels, pills, nasal spray, inhalers, snuff, suppositories, gummies, and other formulations.

In at least one non-limiting embodiment the composition or formulation described herein may include a smokeable, drinkable or edible. In an embodiment it may include infused tobacco with a low-grade nicotine, and/or chewing tobacco, and/or a chewable or other hemp product. Levels of THC in the composition or formulation may be infused at different strengths, including between 5-25 mg in a non-limiting example. The medicinal properties of the terpenes and hemp provide solutions for various ailments as described herein. In one example, the hemp flower may be infused with one or more terpenes described herein. Furthermore, the hemp flower may also, or alternatively be infused with THC at various strengths.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1 includes a chart providing various embodiments of the formulation or composition. Numerous combinations are possible as shown in the chart. The chart of FIG. 1 provides various embodiments in which a smokable, a terpene, THC (synthetic and/or organic) and a delivery system are combined. However, in other embodiments, as otherwise described herein, one or more of these components may be omitted from the formulation. For example, a delivery system may include a smokable and terpenes only. In another embodiment, a smokable and THC may be combined with or without terpenes.

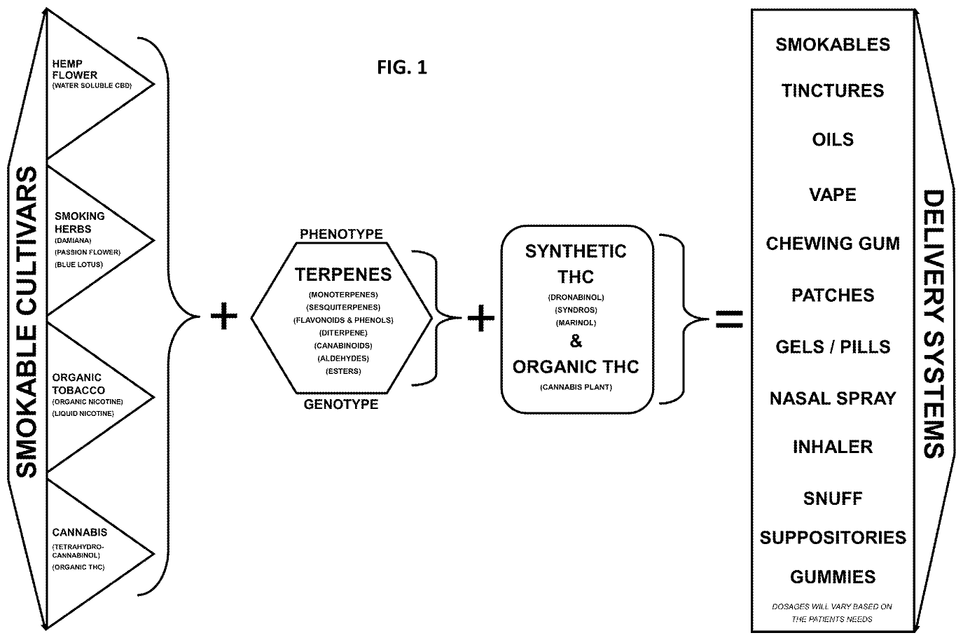

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” The modifier “about” used in connection with a quantity is inclusive of the stated value and has the meaning dictated by the context (e.g., includes the degree of error associated with measurement of the particular quantity). It is to be noted that all ranges disclosed within this specification are inclusive and are independently combinable.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

In one embodiment, a composition is provided including 1) a terpene comprising at least one of: a) a primary terpene; b) a secondary terpene; and c) a tertiary terpene; 2) THC including a) synthetic THC, or b) organic THC, or a combination thereof; and optionally, 3) a natural smokable.

The composition may include a primary terpene including at least one of α-pinene, α-bisabolol, β-pinene, guaiene, guaiol, limonene, myrcene or ocimene. The primary terpene may be provided in an amount of 50% (w/w) or less of the formulation, in one example. The secondary terpene may be provided in an amount 50% (w/w) or less of the formulation, in one example. And the tertiary terpene may be provided in an amount of 50% (w/w) or less of the formulation, in one example.

In one non-limiting embodiment, the primary terpene may be provided in an amount of 50% (w/w), the secondary terpene is provided in an amount of 40% (w/w) and the tertiary terpene is provided in an amount of 10% (w/w) of the composition.

In one example the primary, secondary or tertiary terpene may be provided at 1%-100% by weight of the composition. In another example, the THC may be provided at 1%-100% by weight of the composition.

At least one of the primary terpene, secondary terpene and tertiary terpene may include one or more of the group consisting of: monoterpenes, sesquiterpenes, flavonoids, phenols, diterpenes, cannabinoids, aldehydes, or esters. The synthetic THC may include at least one of: Dronabinol, Syndros, or Marinol. The organic THC may include THC derived from a cannabis plant. Examples of natural smokables described herein may include hemp flower, smoking herbs, tobacco, or cannabis. The hemp flower may include CBD in some non-limiting examples. Smoking herbs described herein may include damiana, passion flower, or blue lotus, or a combination thereof.

The primary, secondary or tertiary terpene may be purified from a natural source, or may be synthetic, in non-limiting examples.

In one non-limiting embodiment, a method of treating a nicotine addiction in a subject is provided including administering to the subject a therapeutically effective amount of the compositions described herein. In one example, the composition includes nicotine. In a further embodiment, the method may include decreasing the percentage by weight of nicotine and increasing the percentage by weight of THC. In some non-limiting examples, the percentage by weight of nicotine may be decreased by the same amount as the increase in percentage by weight of THC over time. The method may further include further decreasing the percentage by weight of nicotine and increasing the percentage by weight of THC.

In the method embodiments described herein, the percentage of nicotine may be decreased by at least 5% and the percentage of THC may be increased by at least 5%.

In the method embodiments described herein, the therapeutically effective amount of the composition may be decreased by at least 10% per administration. In another embodiment, the method may be provided herein, wherein the therapeutically effective amount decreases by at least 10% per week of administration. In still another embodiment, the method may be provided, wherein the composition is administered to a subject daily. In another embodiment, the composition may be administered to the subject twice daily. In still another embodiment, the composition may be administered to the subject weekly or bi-weekly, in non-limiting examples.

In yet another embodiment, a formulation including a terpene, THC, and a delivery system may be provided. In a non-limiting example, the formulation may further include a natural smokable. The delivery system may be selected from the group consisting of: smokables, tinctures, oils, vapes, chewing gum, patches, gels, pills, nasal spray, inhalers, snuff, suppositories, and gummies. The formulation may be provided wherein the THC comprises synthetic THC which may include dronabinol, syndros, or marinol, in non-limiting examples. The formulation may include a terpene selected from the group consisting of: monoterpenes, sesquiterpenes, flavonoids and phenols, diterpene, cannabinoids, aldehydes, and esters. In one example, the formulation may include a natural smokable selected from the group consisting of: organic tobacco, smoking herbs, cannabis or hemp flower, or a combination thereof.

In still another embodiment, a formulation is provided including natural hemp; one or more natural terpenes; and synthetic THC. The formulation may include between 1-25 mg synthetic THC, in one non limiting example.

In at least one embodiment, a delivery system comprising a smokable, for example, may include a composition or formulation as described herein, including at least 5 mg of THC per smokable (or 100 mg THC per pack of smokables). The smokables may be in the form of a cigarette in a non-limiting example. The quantity of nicotine in the composition may include between 0.0001 mg nicotine and 40 mg nicotine in a non-limiting example. However, in a particular, non-limiting example, the quantity of nicotine in the composition include 11 mg, and the amount of the may include 5 mg during a first dosing cycle (wherein the dosing cycle may occur daily for one or more weeks, or weekly, for one or more months). In some examples, a first dosing cycle may include multiple doses per day. In a second dosing cycle, the amount of THC in the composition may be increased by 1 mg, for example, and the amount of nicotine in the composition may be decreased by 1 mg. In the third dosing cycle, the amount of THC in the composition may be further increased by 1 mg, for example, and the amount of nicotine in the composition may be further decreased by 1 mg. In some instances the quantity of each component may be increased or decreased by a greater or lesser amount depending on the side effects and impact to the user. Ultimately the nicotine may be decreased completely, or removed entirely from the composition and the THC could be increased to 16 mg, for example.

It may take approximately 16 packs of cigarettes to achieve a complete nicotine removal from the cigarettes or smokables. This process may be achieved with synthetic THC being combined with the tobacco or combining the tobacco with low dose THC cannabis bud. The same formula, method, and process applies to the terpenes being added to other smoking cultivars.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

1. A composition, comprising:
a. a terpene comprising at least one of:
i. a primary terpene;
ii. a secondary terpene; and
iii. a tertiary terpene;
b. THC comprising:
i. synthetic THC
ii. organic THC; and optionally,
c. a natural smokable.

2. The composition of claim 1, wherein the primary terpene comprises at least one of:
α-pinene, α-bisabolol, β-pinene, guaiene, guaiol, limonene, myrcene or ocimene.

3. The composition of claim 1, wherein the primary terpene is provided in an amount of 50% (w/w) or less of the formulation.

4. The composition of claim 1, wherein the secondary terpene is provided in an amount 50% (w/w) or less of the formulation.

5. The composition of claim 1, wherein the tertiary terpene is provided in an amount of 50% (w/w) or less of the formulation.

6. The composition of claim 1, wherein the primary terpene is provided in an amount of 50% (w/w), the secondary terpene is provided in an amount of 40% (w/w) and the tertiary terpene is provided in an amount of 10% (w/w) of the composition.

7. The composition of claim 1, wherein at least one of the primary terpene, secondary terpene and tertiary terpene comprises one or more of the group consisting of: monoterpenes, sesquiterpenes, flavonoids, phenols, diterpenes, cannabinoids, aldehydes, or esters.

8. The composition of claim 1, wherein the synthetic THC comprises at least one of: Dronabinol, Syndros, or Marinol.

9. The composition of claim 1, wherein organic THC comprises THC derived from a cannabis plant.

10. The composition of claim 1, wherein the natural smokable comprises: hemp flower, smoking herbs, tobacco, or cannabis.

11. (canceled)

12. The composition of claim 1, further comprising between 0.001 mg-40 mg nicotine.

13. A method of treating a nicotine addiction in a subject comprising:
administering to the subject a therapeutically effective amount of the composition of claim 12.

14. The method of claim 13, comprising decreasing the percentage by weight of nicotine and increasing the percentage by weight of THC.

15. The method of claim 14, wherein the percentage by weight of nicotine is decreased in the same amount as the increase in percentage by weight of THC.

16. The method of claim 14, wherein the percentage of nicotine is decreased by at least 5% and the percentage of THC is increased by at least 5%.

17. The method of claim 13, wherein the therapeutically effective amount decreases by at least 10% per administration.

18. The method of claim 14, wherein the therapeutically effective amount decreases by at least 10% per week of administration.

19. The method of claim 12, wherein the composition is administered daily.

20. The method of claim 12, wherein the composition is administered weekly.

21. The composition of claim 1, further comprising a delivery system wherein the delivery system is selected from the group consisting of: smokables, tinctures, oils, vapes, chewing gum, patches, gels, pills, nasal spray, inhalers, snuff, suppositories, and gummies.
